(19) United States
(12) Patent Application Publication
(10) Pub. No.: US 2011/0009837 A1
(43) Pub. Date: Jan. 13, 2011

(54) DEVICE FOR REMOVING BIOLOGICAL MATERIAL

(75) Inventor: Thomas Schreiner, Gingsterweg (DE)

Correspondence Address: F. CHAU & ASSOCIATES, LLC, 130 WOODBURY ROAD, WOODBURY, NY, 11797, US

(73) Assignee: MILTENYI BIOTEC GMBH, Bergisch Gladbach (DE)

(21) Appl. No.: 12/863,487

(22) PCT Filed: Jan. 19, 2009
(86) PCT No.: PCT/DE2009/000051

§ 371 Date: Aug. 26, 2010

(30) Foreign Application Priority Data
Jan. 17, 2008 (DE) 102008004977.8

(51) Int. Cl. A61M 1/00

(52) U.S. Cl. 604/317

(57) ABSTRACT

The invention pertains to a device (1) for removal of biological material from an organism. The device (1) comprises: a removal means (2) for removing biological material from an organism with a redirection means (3) for selecting luminal connection of a removal means (2) with a) a penetration means (6) for penetrating into the organism, b) a reception means (4) for receiving the material from the organism, and c) a reservoir means (12) for storage of a substance. The direction means (3) is configured such that the removals means (2) can form a luminal connection with the reservoir means (12) and with the reception means (4), or with the penetration means (6).

The present invention refers to a device and a method for removing biological material, for example bone marrow or blood from an organism. At the same time, the device allows for sterile processing (for example, through centrifugation) of the biological material in the device.

There is a large number of diseases during which biological material is needed to be removed from the body of a living or dead organism. Amongst them are, for example, diseases of the hematopoietic and lymphatic systems like acute and chronic leukemias, anemias, lymphomas, solid tumors like mammary carcinoma, thyroid carcinoma, malignant melanoma or diseases of different inner organs like malfunctions of the thyroid, kidney insufficiency, liver insufficiency, and others. Such diseases may require the removal of blood, urine, lymph and other body fluids or the removal of bone marrow.

Biological materials usually consist of various components with different characteristics. Blood, for example, consists of different cell types, like leukocytes and erythrocytes and various components like fats and other bio-molecules. The same is true for bone marrow, which also consists of various types of cells, like for example bone cells (osteoblasts and osteoclasts), stroma cells, blood-forming stem cells, cells of the blood-forming system in various stages and cells of the immune system (B- and T-lymphocytes and their progenitors), as well as fat cells. Bone marrow cells are responsible both for formation of blood and of lymphatic cells as well as for the repair of various tissues of the body and of blood vessels.

Bone marrow is a soft mass (so-called spongiosa) with trabeculae that is located in the middle of the surrounding hard bone component (corticalis).

In various diseases of the blood-forming system and also the immune system, a bone marrow transplantation is performed. In most cases, such diseases are solid tumors, leukemia, lymphomas and dysfunctions of blood formation like aplastic anemia, for which bone marrow is used. In order to treat the patient, either autologous (own) stem cell preparation or allogeneic (foreign) stem cell preparations are used. The success of a stem cell transplantation depends on, among other things, how genetically compatible the allogenic preparations of the donor and the recipient are, that is how identical the genes of the human leukocyte antigen (HLA) complexes are. If donor and recipients exhibit too great a difference, severe side effects may occur, such as rejection of the transplant or graft verses host disease (GvHD), which can cause grave problems including the death of the patient.

The success of the transplantation also depends on the quality of the transferred biological material, for example, the blood or the bone marrow. While the withdrawal of blood is relatively unproblematic, the removal of bone marrow is significantly more demanding. Since the removal is painful, it is performed under general or local anesthesia. Moreover, the corticalis needs to be penetrated to reach the bone marrow. This requires, particularly, devices that are suited for penetrating the bone and generating enough room for a hollow needle (cannula) that is big enough not to damage the cells of the bone marrow during removal. This removal is performed either manually by sucking the bone marrow out using a syringe or through the use of pumps. Such devices are known to a person of skill in the art.

Currently, the bone marrow is often removed using a so-called Jamshidi cannula with a syringe. This is a hollow needle with a rounded tip, a trocar (also called mandrin) with a honed, sharp tip and a grip with an adaptor for the syringe (Luer adapter). This multi-part assembled device is manually drilled through the hard bone wall with its sharp tip. Subsequently, the trocar is removed, by which the hollow needle remains by itself in the bone marrow. Then, a syringe is placed on the adapter and a relatively small volume of bone marrow is sucked into the syringe.

In order for the bone marrow not to coagulate, a coagulation inhibiting substance needs to be drawn up Typically, this is not performed in a designated clean-room that fulfills the requirements for producing pharmaceuticals, but at best, in an environment such the operating room with its associated risk of contamination.

Since the volume that can be taken up using a syringe is in most cases is not sufficient for a single transplantation, the syringe must be placed into different areas of the bone to obtain enough bone marrow. The repeated placement of the syringe followed by the emptying of the bone marrow from the syringe into a container increases the danger of contamination through particles and microorganisms in the air. The microorganisms are particularly problematic for the transplant patient, since his immune system has been weakened prior to the transplantation in order that the newly transplanted bone marrow is accepted by the host. Since the patients have to live without leukocytes for at least ten days or with an impaired immune response for four to 50 weeks, each contamination can turn out to be a life-threatening risk for them. The contamination cannot be detected before the administration the bone marrow, because the transplantation is performed within hours after the removal of the bone marrow, that is at a point in time when no results about a possible contamination of the bone marrow to be transplanted is obtainable.

Moreover, it is very cumbersome for the surgeon removing the bone marrow to refill the syringe each time with new coagulation inhibitor and to connect again with the cannula present in the bone marrow in order to continue with the process of bone marrow removal.

### DESCRIPTION OF THE INVENTION

The problems mentioned are solved with the present device for the removal of a biological material from an organism and the processing of the biological material that is capable of flow.

The device according to the invention for removal of biological materials from an organism is in a preferred embodiment also suited for separating the biological samples into at least two of its components.

The device comprises a removal means for removing biological materials from an organism. The removal is performed by generating negative pressure (suction, under-pressure) with respect to the pressure in the organism. The removal device also allows for the release of the material in a reception means of the device of the invention for receiving the materials from the organism and for later processing of the biological sample (for example, through centrifugation etc.). The release, like the reception, is performed through generating of a pressure difference, i.e. by generating a positive pressure with respect to the surroundings.

In one embodiment, in which the device is also suitable for separating the biological sample into at least two of its components, the reception means is preferably configured such that the biological material can be separated into at least two of its components in the reception means, such as through centrifugation. For example, blood in the reception means can be separated through centrifugation of the reception means into plasma and erythrocytes. Materials that are suited for producing such reception means are known to a person of skill in the art.

In one embodiment, a redirection means can be located at the removal means of the device, for example, by use of a tube connection. Alternatively, the redirection means can be integrally implemented as one-piece with the removal means. The one-pieced implementation has the advantage of a lower risk of contamination. This redirection means serves the purpose of selecting a luminal connection of the removal means with the mentioned reception means, a penetration means for penetration into the organism, or a reservoir means for storage of a substance, in particular for storing an anti-coagulant. This selection of the luminal connection is afforded through particular functional positions of the redirections means.

The redirection means such that the removal means in each of its possible functional positions can be luminally connected either only with the reservoir means and the reception means, or only with the penetration means. Alternatively, the redirection means can be brought into a position in which the removal means can be uncoupled from the other parts of the device. In this position of the redirection means, only reservoir means, the reception means and the penetration means are luminally connected to each other (no functional position). In the position of the redirection means, the removal means can, for example, be removed and replaced.

The device of the invention constitutes in one embodiment a system that is open on one side, which is only open to the surroundings via the penetration means (and possibly at the redirection means). Thereby, a sterile system is provided for and the danger of contamination is lowered. Preferably, the device is made of a material that is impermeable to microorganisms. In a further embodiment, at least one connection means (e.g. in the form of a Luer connector) is present for connecting at least one further component.

The device of the invention can be used for removal of a biological material from an organism and processing of the biological material that is capable of flow. In particular, the device can be used for all body fluids like blood, urine, lymph or liquor, but also for tissues, cells or fractions or components thereof. The device is particularly suitable for the removal of bone marrow. In a preferred embodiment, the device of the invention is accordingly a bone marrow removal device and a bone marrow processing device; in another preferred embodiment, the device of the invention is a blood-removal and a blood-processing device. Organisms from which biological material can be removed using the device are, in particular, mammals, preferably humans.

Preferably, the redirection means is located between the removal means and the penetration means. It is also preferred that the redirection means is located between the reservoir means and the reception means. Accordingly, in a preferred embodiment, the redirection means that is connected to the removal means is luminally connected via a conduit means on a first side of the penetration means, on a second side of the reservoir means and on a third side to the reception means.

In order to determine the direction of flow of the biological material, the device comprises, in a preferred embodiment, in the conduit means between the reservoir means and the redirection means, a first valve (e.g. a first unidirectional restriction valve) that is placed such that the stored substance can flow only from the reservoir means in the direction to the redirection means, but cannot flow in the opposite direction.

Similarly, in another preferred embodiment, which may also comprise the first valve, a second valve may be located between the redirection means and the reception means, e.g. a second unidirectional restrictor valve in the conduit means, such that the removed biological material and/or the stored substance in the reservoir means may flow from the redirection means only in the direction towards the reception means.

The presence of such valves is advantageous in particular when the device serves for removing and possibly processing (separating) bone marrow or is used to create high pressure for this purpose, as the risk is high that a substance within the device, such as biological material or anti-coagulant present in the reservoir means may be moved in the lumen in an unwanted direction in the presence of such high pressure.

In order to ensure sufficient sterility, at least one filter for filtering microorganisms and/or pieces of material is preferably located in the conduit means.

In particular, the present combination of removal means, redirections means, penetration means, reservoir means and reception means with at least one valve and at least one filter in a single device allows for the contamination-free removal and also processing of biological material such that the device can, for example, be used in operating rooms. Therefore, the use of certified clean-rooms for processing (e.g. separating) the biological material is no longer necessary. Separated components of the biological material may, for example, after manipulation such as the addition of at least one medicament, may be added to the organism from which it was originally removed or to another organism.

In a further embodiment of the device, the removal means is a syringe or pump with which the necessary pressure may be created for transporting the biological material and/or the stored substance in the conduit means. The redirection means is preferably a three-way stop cock. For increasing ease of use of the device, the conduit means is preferably a flexible tube. As a penetration means, a cannula, in particular a Jamshidi cannula is used.

In a preferred embodiment, the reception means for the removed biological material comprises one or several containers, in particular three containers, preferably in the from of bags. This has the advantage that, after removing the biological material, a processing of the material can take place in the reception means itself without the necessity for transferring the biological material. Before processing, the reception means may be detached from the rest of the device using a connector located in the conduit means. In an alternative embodiment, the detachment occurs by melting off the afferent conduit means, whereby a portion of the conduit means that leads towards the reception means is at the same time sealed in a contamination-free manner. The processing is preferably centrifugation, and this is why the reception means is preferably made of a material suited for centrifugation.

The substance stored in the reservoir means is preferably an anti-coagulant, like a heparin-containing solution or a CPD (citrate, phosphate, dextrose) solution.

Preferably, the device is designed to be a disposable article for one-time use. Thus, it need not be cleaned and sterilized after use. The device should be made from a material that can be sterilized (for example through irradiation with gamma rays or through chemical sterilization).

The problem underlying the present invention is also solved by the use of a device described herein for removal of a biological sample from an organism.

The problem underlying the present invention is also solved by a method for removal of biological material from an organism, comprising the removal of biological materials from an organism using a device as described herein.

The method comprises the charging or filling of the removal means and/or the reception means of the device with the substance stored in the reservoir means, in particular in the form of an anti-coagulant. In further steps, an organism is penetrated with a penetration means (for example in the form of a cannula), biological material is removed from the organism into the removal means and the material is then transferred from the removal means through creation of a pressure difference into the reception means.

The problem underlying the present invention is also solved by a method for separating a biological sample from an organism into at least two of its components. This method comprises the method for removing a biological sample from an organism as described above using the present device. Moreover, it comprises the step of separating the biological material in the reception means into at least two of its components for further processing, in particular through centrifugation. Blood can, for example, be separated into serum and cellular components. In cases where the reception means of the device comprises several containers, in further steps of the method, the serum that has been separated from the blood may be transferred into a further container of the reception means by applying pressure on the container that contains the serum and the cellular components.

In a preferred embodiment, the invention does not refer to a method for surgical or therapeutic treatment of the human or animal body or to a diagnostic method that is practiced on the human or animal body.

### FIGURES

The figures show:

FIG. 1 a device wherein the reception means comprises three bags that are connected via a central Y-piece, wherein a Luer lock is located below the Y-piece;

FIG. 2 a device in which the reception means comprises three bags, which are connected via a central Y-piece, wherein a 200 μm filter is located below the Y-piece;

FIG. 3 a device in which the reception means comprises a collection bag and two fraction bags connected thereto;

FIG. 4 a device in which the reception means comprises a collection bag and connected thereto two fraction bags, wherein a 200 μm filter is located in the conduit means;

FIGS. 5 to 23 the functions of the device of the invention and the method of its use.

The drawings of the figures are described in more detail below.

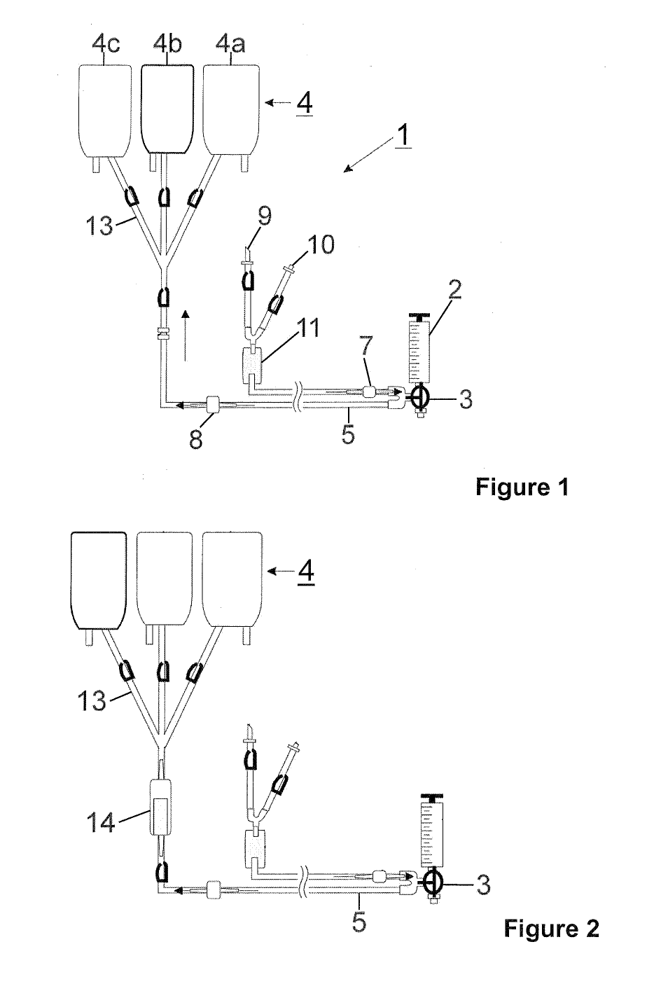

### FIG. 1

The removal device 1 shown comprises a removal means in form of a (piston-) syringe 2, a redirection means in the form of a multi-way cock (here a three-way cock) 3, a reception means 4 in the form of three bags 4a, 4b, 4c and a conduit means 5 in form of a tube.

The conduit means 5 connects the three-way cock 3 on the one hand luminally with the reception means 4 in the form of three bag 4a, 4b, 4c and on the other hand luminally with the reservoir means (not shown). In the embodiment of the device 1 shown, the removal means in form of a syringe 2 is located immediately at the three-way cock 3, which could also be implemented through a conduit means 5.

A penetration means in form of a Jamshidi cannula (not shown) can be connected via a Luer lock at the three-way cock 3 of the device 1. A reservoir means (not shown) in the form of a container for the anti-coagulant can also be connected with the conduit means 5 of the device 1.

The syringe 2 is connected to the three-way cock 3 and the anti-coagulant substance and the bag 4 are connected through the conduit means 5, through which a repeated removal of an old syringe and the attachment of a new syringe can be avoided. As an anti-coagulant substance, un-fractionated heparin could, for example, be used, that is suitable from intravenous application.

When using the device, the (not shown) cannula is introduced for example into the bone marrow of a patient. First, anti-coagulant solution is sucked out of the reservoir means 12 into the syringe 2, which is connected to the removal device 1 by one of two integrated adapters (e.g. in form of a spike or female Luer), and is functionally closed from the surroundings due to integration of a first bacteria-impermeable filter 11. Thereby, the entrance of germs when sucking the anti-coagulant solution is eliminated.

Through the syringe 2, which is attached at the three-way cock 3, the biological material can be sucked up and thereby reaches the syringe 2. When the syringe 2 is filled, the three-way cock 3 is switched and thereby the connection to the reception means 4 is opened. When the syringe 2 is emptied by applying pressure, the biological material flows into the reception means 4 without exiting the closed system of the device 1 or without the need to attach another syringe 2. By incorporating two unidirectional restrictor valves 7, 8 into the conduit means 5, it is insured that the biological material cannot be transferred into the wrong bag 4a, 4b, 4c.

The process of removing biological material through filling and emptying the removal means 2 can be repeated as many times as wished without opening the device 1 at any location. Thereby, the entrance of harmful germs can be avoided. Only the connection between the three-way cock 3 and the Jamshidi cannula can be opened when the physician performing the puncturing would like to penetrate the hard bone shell using the trocar in another location.

Biological material can be any liquid or semi-solid biological substance, like blood, urine, lymph and other body fluids, bone marrow or fat. Bone morrow is preferred.

The conduit means 5 of the device 1 of the invention is preferably made of a flexible material. Preferably, tubes 5 are used with an outer diameter of 3 mm to 6 mm, preferably 4.1 mm and with an inner diameter of 1.5 mm to 4.5 mm, preferably 2.5 mm and a lumen of 0.5 mm to 10 mm, preferably 2 mm to 5 mm Preferred materials are, for example, plastic, polyethylene and/or materials that are translucent and/or colorless and non-toxic.

The components of the redirection means 3 need to be suitable for sucking up, collecting and releasing biological material either manually or using an instrument. Preferably, this is a syringe 2 with a volume of 2 ml to 20 ml, preferably 10 ml volume and with a simple or preferably a multiple rubber seal, like for example polypropylene syringes with polyisoprene seal and Luer lock connector. The solid connection between the removal means 2 for biological material and the three-way cock 3 prevents the introduction of particle-contaminated air as well as harmful germs.

In the embodiment shown, the device 1 comprises two entry points to the conduit means 5, namely a spike 9 and a female Luer connector 10. In this embodiment, the components of a first entry point for the reservoir means 12 (e.g. in form an infusion bag), which leads to the three-way cock that is connected to the removal means 2 comprises:
a spike 9 with an inner diameter of 1 mm to 5 mm, preferably 3 mm, and an outer diameter of 2 mm to 8 mm, preferably 4.1 mm, or with a diameter that fits the tube used for connecting to the infusion bag, as well as
a female Luer connector 10 (preferably acrylic with a diameter of 3.1 mm to 3.4 mm (inner diameter) or fitting to the tube size used) for connecting to a syringe,
a first filter 11 for preventing the entrance of bacteria via the sucked-up liquid, as well as
a first unidirectional valve 7, such that the direction of flow is determined in the direction to the three-way cock 3 and that no biological material can flow in the direction of the spike 9 and/or the female Luer connector 10.

The first filter 11 has a pore size of 0.05 μm to 0.2 μm, preferably of 0.2 μm. Preferably, the filter 11 can catch harmful germs (like bacteria and fungi), particles etc., such that a contamination of the device 1 is prevented. Further, the filter 11 is preferably configured such that it can carry out a self-priming (wetting the surface with liquid and replacing the air in the housing). Further preferred, it is has high flow rate (more than 5 ml per minute to 10 ml per minute).

The first (unidirectional restrictor) valve 7 is preferably located between the bacterial filter 11 and the redirection means (e.g. a three-way cock 3). The valve 7 spares the user from having to switch this three-way cock 3 so that the use of the device is particularly easy compared to the methods known in the state of the art, which require a repeated positioning and moving and mounting of the syringe, or that harbor a higher risk for mistakes during use when a second three-way cock is present.

The first (unidirectional restrictor) valve 7 has preferably a high opening pressure of 0.2 to 5 PSI (Pascal per square inch) (14-345 mbar), preferably 1.5 PSI (103 mbar) (e.g. Qosina check-valve, diameter 0.130 to 0.138 (outer diameter), opening pressure (cracking pressure) 0.5 to 5.0 PSI (34-340 mbar), preferably 1 to 2 PSI, further preferably 1.5 PSI (103 mbar) (housing acrylic, valves out of silicon)) in order to prevent the unintentional overflow of the anti-coagulant in case of an elevated position, for example at an infusion stand, through the valve. If the valve 7 opens already at low pressure, the anti-coagulant flows by itself into the reception container. This is prevented through the elevated opening pressure.

The spike 9 is preferably connected with the reservoir means 12 with a ready-to-use solution of CPD (citrate, phosphate and dextrose), preferably with a volume of 250 ml CPD, containing citrate, phosphate and dextrose.

In the embodiment shown, the female Luer connector 10 can be connected with a syringe 2 with a male Luer connector, that may contain a mixture of anti-coagulant substance, preferably heparin (e.g. un-fractionated heparin, suitable for intravenous applications, 500 IE/ml) and either NaCl 0.9% or CPD. The syringe 2 has preferably a volume of 10 ml to 50 ml.

A further entry point located at the three-way cock 3 comprises a connection to the reception means 4. A tube of 2 cm to 5 cm length, preferably of 3 cm with an inner diameter of 2.5 mm is integrated between the three-way cock 3 and a two armed Y-piece 18 of the distribution of the two efferent and afferent tubes to improve the flexibility and ease of use of the sucking device (three-way cock 3 and syringe 2). The length of the tube segment of the conduit means 5 is to be provided such that the amount of anti-coagulant sucked up in each cycle is at least twice as large as the dead volume in the tube segment. In a preferred embodiment, the dead volume of the tube segment is 0.5 ml when removing 1 ml of anti-coagulant substance.

In a preferred embodiment, the tube segment of the conduit system 5 between the reception means 4 and the three-way cock 3 comprises a second unidirectional restrictor valve 8 (duck-bill check valve) as well as a Luer lock connector 16 through which a connection to a portion of the conduit system can be formed, which is connected to the reception means 4 (here in the form of three reception containers 4a, 4b, 4c that are connected to each other). The containers 4a, 4b, 4c are preferably bags, that may be made from synthetic material like PVC. The volumes of the bags can be 100 ml to 1000 ml, preferably 300 ml.

The duckbill check valve 8 can consist for example of polycarbonate (housing) and silicon (valve) and allows through its shape the passage of bone splinters without loosing its ability for closing up, whereby its size matches the connection or tube system, preferably 0.130 inches (outer diameter), with an opening pressure of 0.05 to 0.5 PSI (3,4 to 34 mbar), preferably 0.112 PCI (7.7 mbar) (0.05 to 0.5 PSI tolerable range). Both the valves 7, 8 in the conduit means have to be configured such (functionally harmonized) that the second valve 8 does not open when applying negative pressure to the first valve 7 by pulling the syringe 2 and vice versa: when the syringe is emptied again into the system via the second valve 8, the first valve 7 may not open. This is ensured in the device 1 of the invention.

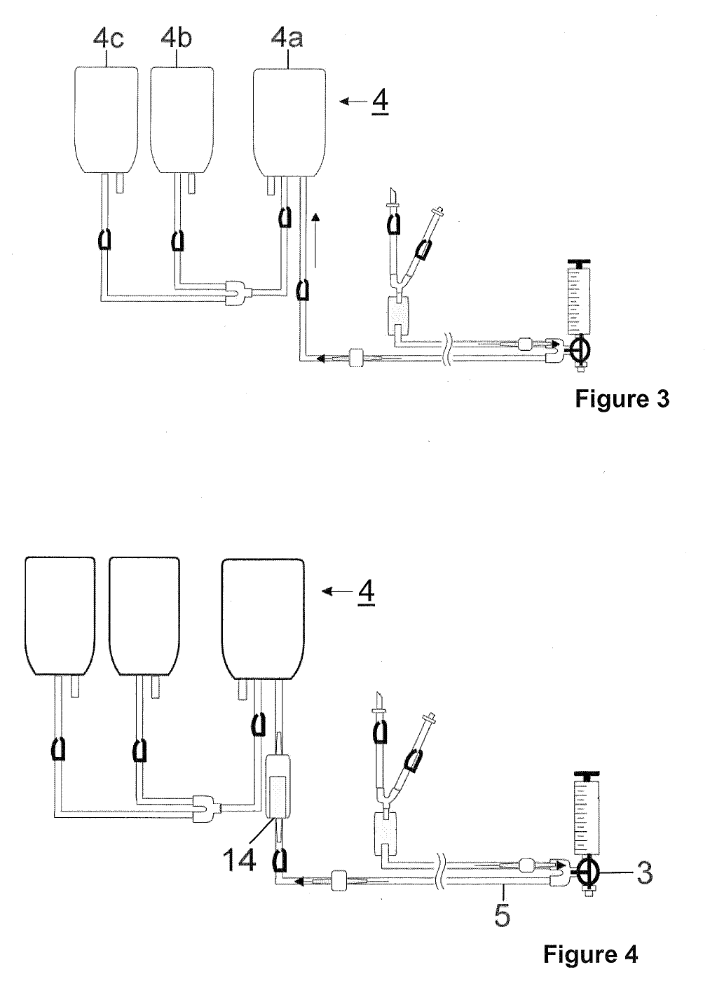

The Luer lock connector 16 is positioned between the second unidirectional restrictor valve 8 and the containers 4a, 4b, 4c and allows the user to remove the receptions means 4 from the rest of the device 1 as well as the implementation of the reception means 4 either in form of a first reception container 4a that is connected with further reception containers 4b, 4c (see FIGS. 3 and 4) or in the form of three containers 4a, 4b, 4c (as shown here). The male Luer lock connector 16 preferably has a diameter of 2 mm to 4 mm, preferably 3.1 mm to 3.4 mm (inner diameter) made out of acrylic or depending on the tube used. The male connector has an inner diameter of 3.1 mm to 3.4 mm or dependent on the tube used and is preferably made out of acrylic.

In the embodiment shown, the conduit means 5 comprises a three-armed Y-piece between the redirection means 3 and the reception means 4, such that the conduit means 5 in the example shown branches into three branches, wherein each branch leads to a bag 4a, 4b, 4c.

Preferably, spike connectors are used at the reception bags 4a, 4b, 4c to allow for the addition or the removal of liquid into or out of all reception bags 4a, 4b, 4c or to improve the flexibility of separating the fractions through a connection with further receptions bags 4a, 4b, 4c. This addition of liquid may be the addition of medicaments or substances that have an influence on the components of the body fluid. Further, samples from the body fluid can be obtained before and after the separation into fractions through centrifugation. Through the connection of several reception bags, it becomes possible to separate the body fluid into several fractions and/or to process them separately in several containers.

The reception containers (bags) of the reception means 4 are preferably configured such that the body fluid can be separated through centrifugation into single fractions. For this, bags of DEPC-free synthetic material are preferred.

The conduit means 5 leading to the bags 4a, 4b, 4c of the reception means 4 can each comprise a clamp 15 for manually controlling the out flow and the transfer of liquid after the centrifugation.

As body fluids, the following fluids can be used: bone marrow, wherein small bone splinters and pieces can may optionally be removed through filtration; blood, wherein in most cases a filtration will only be necessary before back-transfer into the organism; fat tissue, wherein in tubes, three-way cock, filter and valves should be configured such that no blockage from fat vacuoles occurs.

The entire device 1 can preferably be sterilized in order to allow for the removed body fluid or single components of the body fluids to be processed in the absence of germs or to be injected back into the body.

The embodiments shown in the FIGS. 2 to 4 in most aspects are identical to the embodiment described above, so that in the following, only those differences with respect to the embodiment described above are pointed out.
FIG. 2

In the embodiment shown, a second filter 14 is located in the conduit means 5 between three-way cock 3 and the suspension means, which has a preferred pore size of 40 μm to 500 μm, most preferred between 150 μm to 220 μm, further preferred of 200 μm

The second filter 14 serves to remove pieces, clumps, cell aggregations, blood fat, bone pieces or bone splinters and other components. The filtration is useful when the body fluid is to be re-transferred into the body without separation of larger particles. The second filter 14 should be used when the removed biological material that has been transferred into the reception means 4 is to be processed further, such as through centrifugation.

In the embodiment shown, the first bag 4a of the reception means 4 comprises a conduit means 5 for the outflow of biological material from the first bag into at least another, here into a second further bag 4b, 4c. The efferent conduit means 5 that comes out of the drainage of a first reception bag 4a of the reception means, is connected to a Y-piece 13 such that the conduit means 5 branches and one branch of the conduit means 5 leads to the second bag 4b and another branch of the conduit means 5 leads to the third bag 4c. Other embodiments with practically any number of bags are possible.
FIG. 3

The shown embodiment of device 1 comprises a reception means 4 with a first reception bag 4a that is luminally connected with at least one reception bag, in the example shown with two reception bags (a second bag 4b and a third bag 4c). In this embodiment, it is ensured that the body fluid in the tube portion leading to the first bag 4a of the reception means 4 is not remixed after a separation of the fractions with the separated fractions in the second bag 4b and the third bag 4c.

In this embodiment, both the filtration of particles from the body fluid and the prevention of the mixing between the collected and the separated body fluid is possible.

### FIG. 4

The shown embodiment of device 1 is similar to the one shown in FIG. 3 and further comprises a second filter 14 that is located in the conduit means 5 between the reception means 4 and the redirection means 3. The second filter 14 has already been described in the context of FIG. 2.

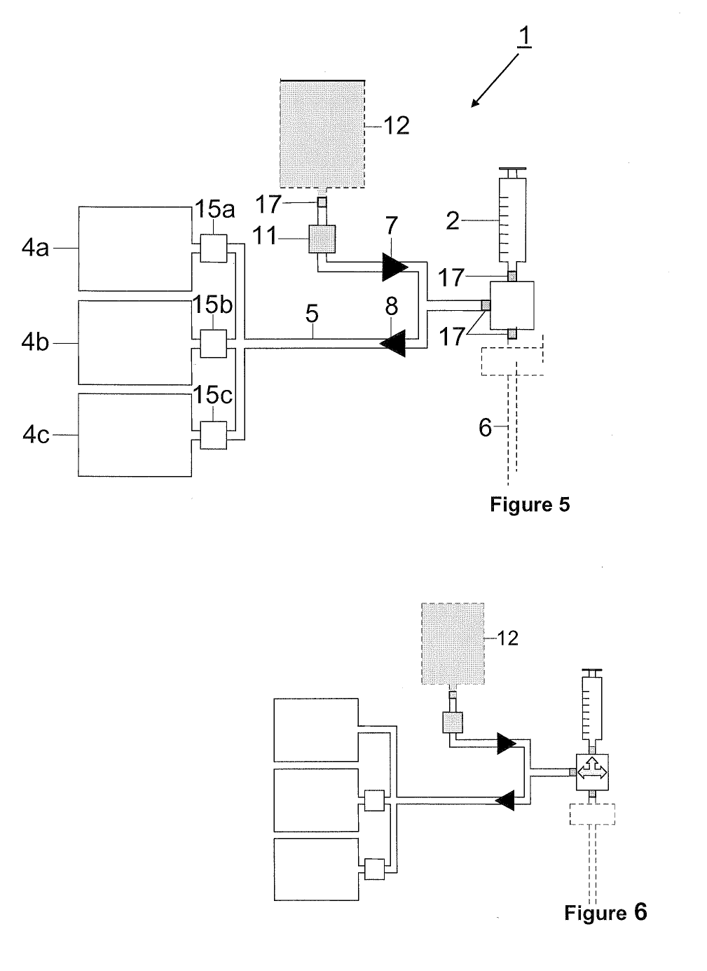

### FIGS. 5 to 23

In FIGS. 5 to 23 the device 1 is described regarding its function.

As shown in FIG. 5, the device 1 comprises a redirection means 3 in the form of a three-way cock, which comprises three connection means 17 in the form of a Luer Lock or a spike. The three-way cock is luminally connected via a first connection means 17 to a removal means 2 in the form of a syringe. Through a second connection means 17, the three-way cock is luminally connected to a penetration means 6 in the form of a Jamshidi cannula and using a third connection means, which is connected to a conduit means 5 in the form of a flexible tube. The tube 5 comprises a first branching wherein a first branch of the tube 5 is connected to a storage means in form of infusion bag containing anticoagulant. The other branch of the tube 5 leads to a reception means 4 in the shape of three bags 4a, 4b, 4c, wherein the conduit means 5 comprises a branching such that to each bag a single branch of the tube 5 is leading. In a portion of the tube 5, shortly before the tube reaches the bag 4a, 4b, 4c, a clamp 15a, 15b, 15c is located in each case, such that the inflow of the tube 5 into the bag 4a, 4b, 4c can be prevented.

In the drawing shown in FIG. 6, the device 1 is in an operational position. In particular, the reservoir means 12 has been charged with anticoagulant such that the biological material to be removed, in particular bone marrow, is prevented from coagulating.

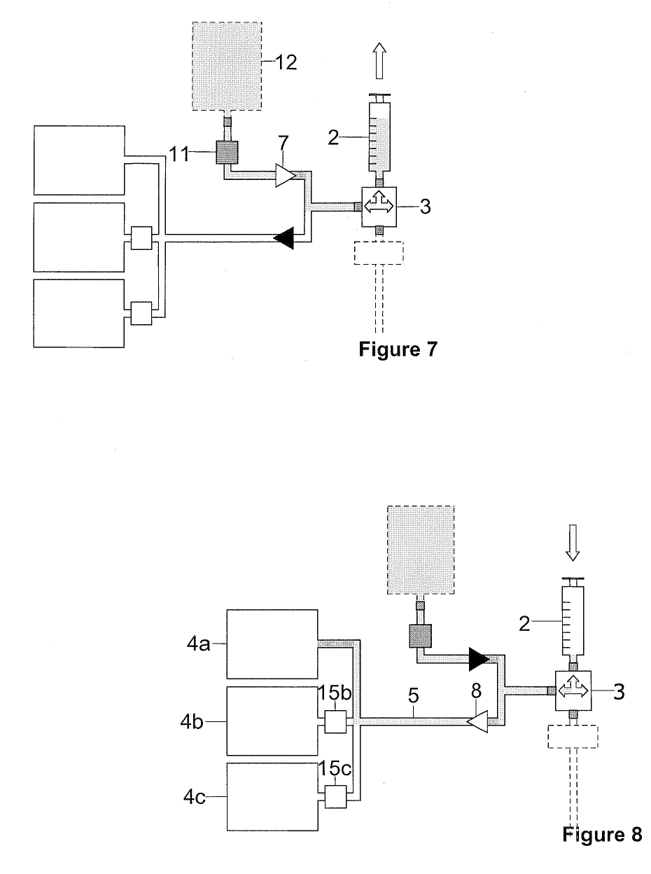

When using the device 1, the three-way cock 3 is initially in a first functional position in which the reservoir means 12 is luminally connected with the syringe 2. By pulling out the piston of the syringe 2, a negative pressure is created in the syringe 2, such that the anticoagulant flows from the infusion bag 12 through the conduit means 5 via the three-way cock 3 into the syringe 2. For this purpose, a first filter is located in the conduit means 5 for filtering pieces of material or microorganisms between the infusion bag 12 and the syringe 2. Further, the tube 5 comprises a first plug valve 17 that defines the flow-direction of the anticoagulant from the infusion bag 12 to the direction of the three-way cock 3 (FIG. 7). An open valve is represented in the figures as a white triangle, whereas closed valve are depicted as black triangles.

A second plug valve 8 is located in the branch of the tube 5 leading to the bags 4a, 4b, 4c; such that when the piston is pressed into the syringe 2, it allows for the passage of the anti coagulant substance in tube 5 into bags 4a, 4b, 4c, while the position of the three-way cock remains unchanged. In the example shown here, the second bag 4b and the third bag 4c are each closed by a second clamp 15b and a third clamp 15c, such that the anti-coagulant can only flow into the first bag 4a (FIG. 8).

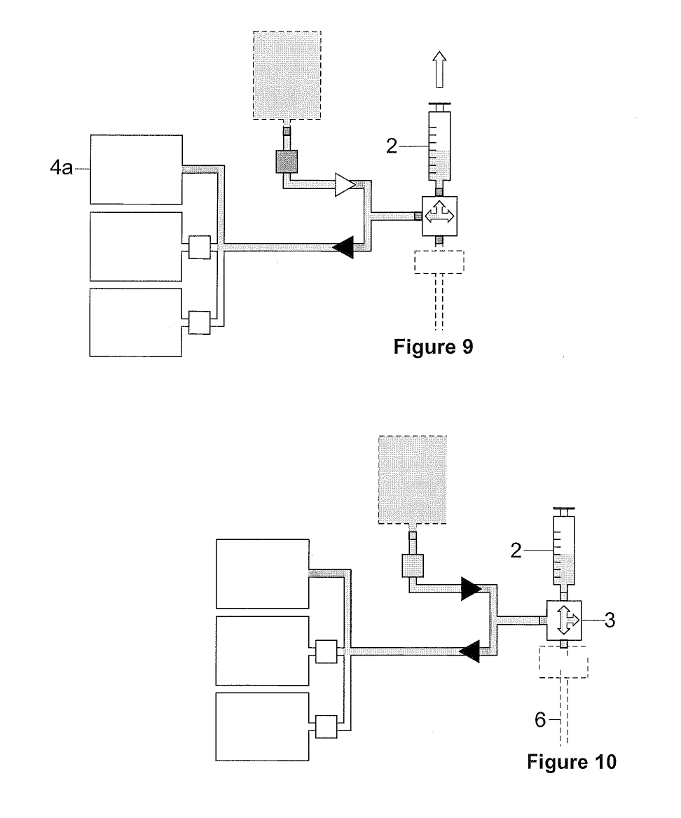

If the volume of the syringe 2 is not sufficient for filling the first bag 4a, the syringe 2 can be refilled by pulling out the piston again. In the example shown here, the syringe 2 is charged with anti-coagulant (FIG. 9).

Now, the cannula 6 can be introduced into the anatomical structure of the organism of interest to begin removing the designated biological material. For this purpose, the three-way cock is brought into a further functional position at which the syringe 2 is luminally connected to the cannula 6 (FIG. 10).

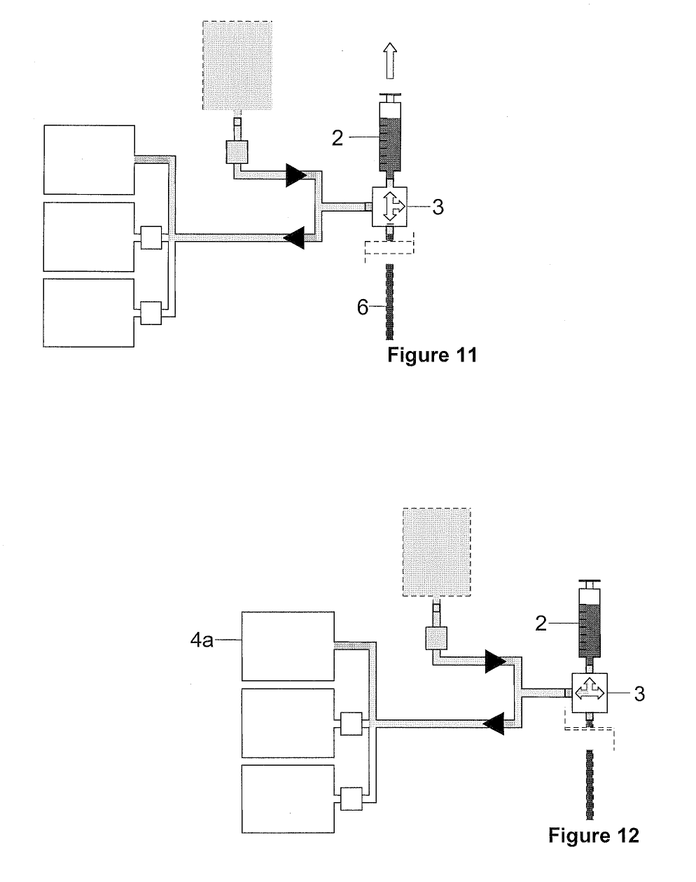

By further pulling out the piston of the syringe 2, a pressure difference with respect to the target tissue is built up such that the biological material that was removed flows through the penetration means 6 via the three-way cock 3 into the syringe 2 (FIG. 11).

After the syringe 2 has been filled with biological material, the three-way cock 3 is moved into the first functional position in which the syringe 2 is luminally connected with the infusion bag 12 and the reception bag 4a (FIG. 12).

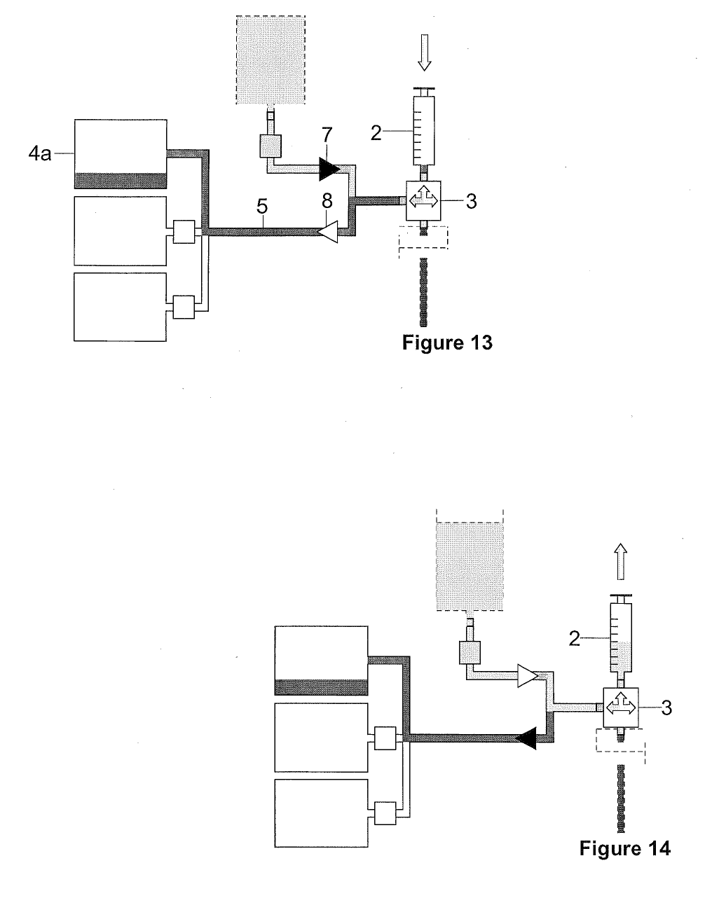

By pressing down the piston of the syringe 2, the biological material flows from the syringe 2 via the three-way cock 3 and the tube 5 into the first reception bag 4a. The first plug valve 7 and the second plug valve 8 in the tube 5 prevent the removed biological material from flowing into the infusion bag 12 (FIG. 13).

Due to the presence of the first plug valve 7 and the second plug valve 8, it is also possible that the syringe 2 can be refilled with anti-coagulant by pulling out the piston in a further step without altering the position of the three-way cock (FIG. 14).

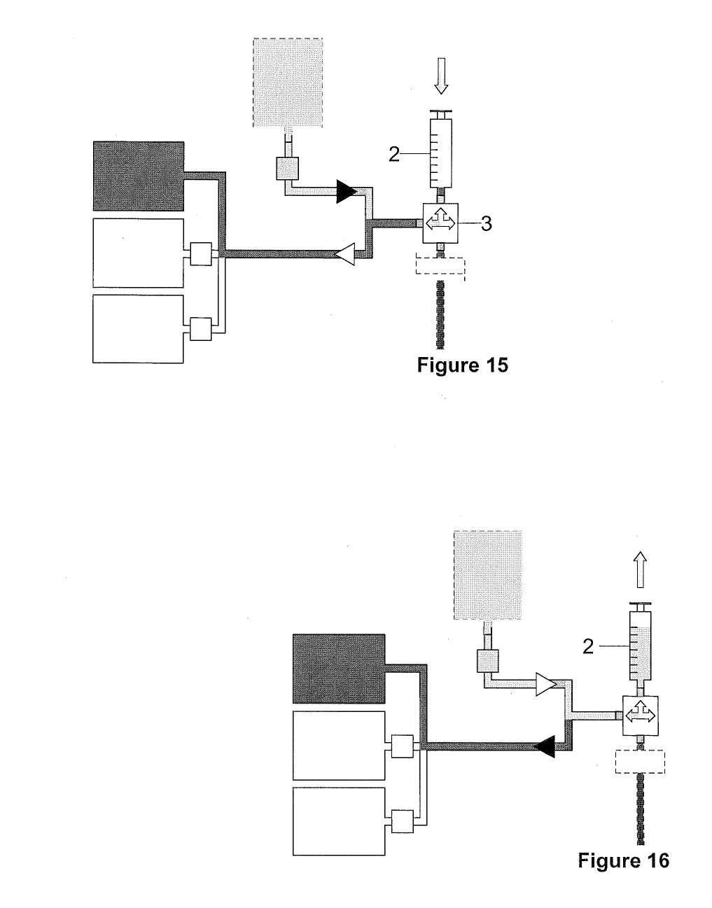

After charging the syringe 2 with anti-coagulant, this anti-coagulant can be moved into the first bag 4a by pressing it out of the syringe without altering the position of the three-way cock (FIG. 15).

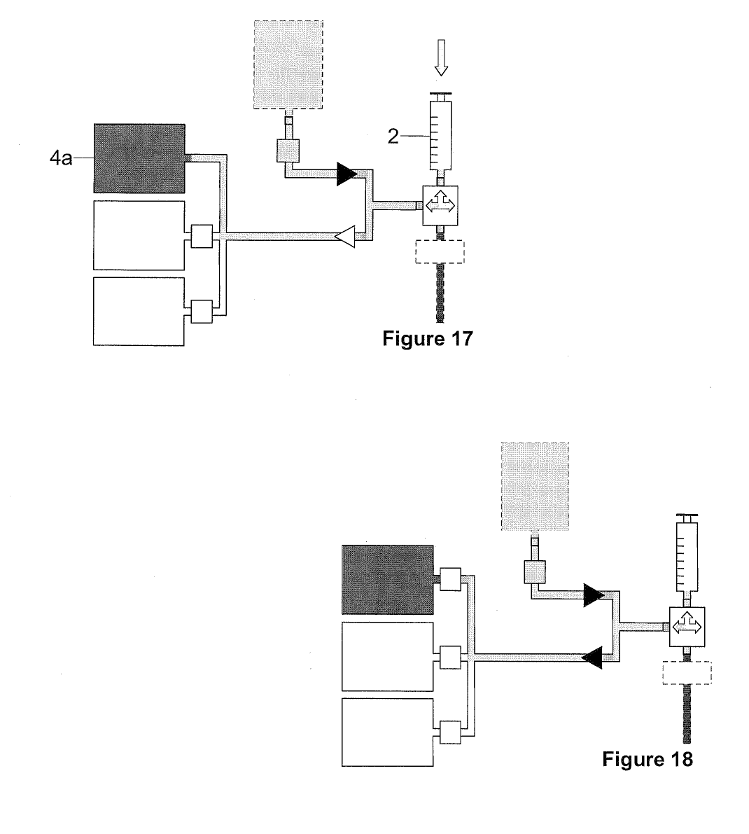

In FIG. 16, the repeated charging of the syringe 2 with anti-coagulant is shown. FIG. 17 shows the transfer of the anti-coagulant into the first bag 4a, as described above. After the removal of the biological material from the organism, the device 1 can be in the position shown in FIG. 18.

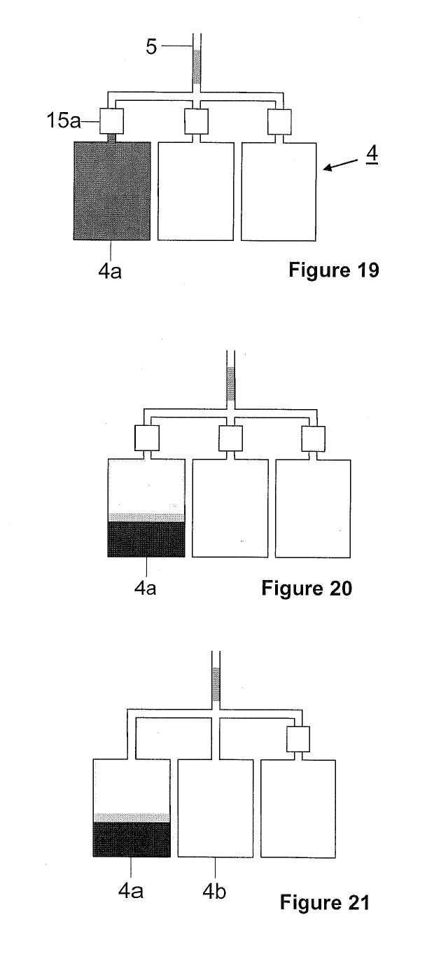

For further processing of the removed biological material, the reception means 3 can be removed through melting off the tube 5 at a suitable position. The tube supply 5 to the first bag 4a is closed prior to the further processing of the removed biological material with a first clamp 15a (FIG. 19).

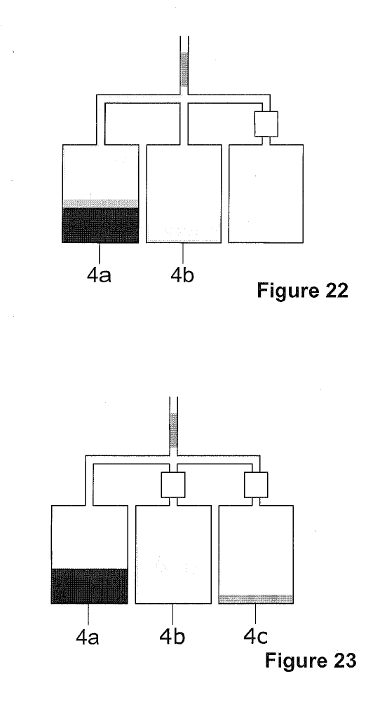

The reception means 4 of the device 1 can, for example, be centrifuged after its removal, wherein for example cellular and liquid components of the biological sample are separated from each other. These components of the biological material are depicted in FIG. 20 in the first bag 4a by different shades of grey. For separating the components of the biological material, the first clamp 15a and the second clamp 15b are opened in this example, such that the first bag 4a is luminally connected with the second bag 4b via the tube 5 (FIG. 21). By applying pressure to the first bag 4a, the component of the biological material forming an upper layer can be transferred to the tube 5 from the first bag 4a into the second bag 4b (FIG. 22). The separation of the biological material may be done using different bags 4a, 4b, 4c and can also be performed through multi-step processing, for example through several centrifugation steps (FIG. 23).

The individual bags 4a, 4b, 4c can now be separated from the other components of the device 1, for example through melting off, or the separated biological material can be removed from the bag by puncturing it with a cannula.

### REFERENCE NUMBERS

1 Device (removal device)
2 Removal means
3 Redirection means
4 Reception means
5 Conduit means
6 Penetration means
7 First plug valve
8 Second plug valve
9 Spike
10 Female Luer Connector
11 First filter
12 Reservoir means
13 Y-piece, three-armed
14 Second filter
15 Clamp
16 Male Luer Lock Connector
17 Connection means
18 Y-piece, two-armed

1-23. (canceled)

24. A device for removal of biological material from an organism, comprising:
a redirection means for selecting a luminal connection of a removal means for removing the biological material from the organism;
a penetration means for penetrating into the organism;
a reception means for receiving the biological material from the organism; and
a reservoir means for storage of a substance,
wherein the redirection means is configured such that the removal means can form the luminal connection with the reservoir means and with the reception means, or with the penetration means.

25. The device according to claim 24, wherein the luminal connection is realized in part by a conduit means.

26. The device according to claim 24, wherein the redirection means is located between the removal means and the penetration means.

27. The device according to claim 24, wherein the redirection means is located between the reservoir means and the reception means.

28. The device according to claim 25, wherein a first valve is located in the conduit means between the reservoir means and the redirection means, wherein the first valve is configured such that the biological material can only flow from the reservoir means towards the redirection means.

29. The device according to claim 28, wherein a second valve is located in the conduit means between the redirection and the reception means, wherein the second valve is configured such that the biological material can only flow in the direction from the redirection means towards the reception means.

30. The device according to claim 29, wherein a filter for filtering at least one of microorganisms or pieces of biological material is located in the conduit means.

31. The device according to claim 24, wherein the removal means is a syringe.

32. The device according to claim 24, wherein the redirection means is a multi-way cock.

33. The device according to claim 32, wherein the redirection means is a three-way cock.

34. The device according the claim 25, wherein the conduit means is a tube.

35. The device according to claim 24, wherein the penetration means is a hollow needle.

36. The device according to claim 24, wherein the reception means comprises one or more than one container.

37. The device according to claim 36, wherein the reception means is configured such that the biological material can be separated into at least two of its components in the reception means.

38. The device according to claim 36, wherein the reception means includes at least one of a flexible, a centrifugable, a transparent or a sterilizable material.

39. The device according to claim 24, wherein the substance that is stored in the reservoir means is an anti-coagulant.

40. The device according to claim 24, wherein the device is disposable.

41. The device according to claim 24, wherein the device can be sterilized.

42. The device according to claim 24, wherein the device separates the biological material into at least two of its components.

43. A method for removing biological material from an organism, comprising:
selecting with a redirection means a luminal connection of a removal means for removing the biological material from the organism;
penetrating into the organism with a penetration means;
receiving the biological material from the organism with a reception means; and
storing a substance in a reservoir means,
wherein the redirection means is configured such that the removal means can form the luminal connection with the reservoir means and with the reception means, or with the penetration means.

44. The method according to claim 43, further comprising charging at least one of the removal means or the reception means with the substance stored in the reservoir means.

45. The method according to claim 43, further comprising:
removing the biological material from the organism into the removal means; and
transferring the biological material from the removal means into the reception means.

46. The method according to claim 45, further comprising:
separating the biological material in the reception means into at least two of its components.

47. The method according to claim 46, wherein separating is performed using centrifugation.

48. The method according to claim 43, further comprising:
removing the biological material from the organism into the reception means; and
separating the biological material in the reception means into at least two of its components, wherein separating is performed using centrifugation.
